(19) United States
(12) Patent Application Publication
(10) Pub. No.: US 2024/0298928 A1
(43) Pub. Date: Sep. 12, 2024

(54) Optimized Acoustic Chirp based on in-vivo BM-delays in human

(71) Applicant: MED-EL Elektromedizinische Geraete GmbH, Innsbruck (AT)

(72) Inventors: Marek Polak, Innsbruck (AT); Giacomo Mandruzzato, Innsbruck (AT)

(21) Appl. No.: 18/273,650

(22) PCT Filed: Jan. 25, 2022
(86) PCT No.: PCT/US2022/013682

§ 371 Date: Jul. 21, 2023

(30) Foreign Application Priority Data
Jan. 25, 2021 (EP) 21153362.5

(51) Int. Cl. A61B 5/12

(52) U.S. Cl. CPC A61B 5/125

(57) ABSTRACT

A system and method of providing an acoustic stimulus for a human subject so as to evoke an auditory response is presented. The method includes creating a chirp signal, wherein creating the chirp signal includes adding a plurality of frequency signals, each frequency signal delayed within the chirp signal based on its associated frequency specific basilar membrane delay determined as a function of measured in vivo frequency specific basilar membrane delays.

### CROSS-REFERENCE TO RELATED APPLICATIONS

This application claims priority from European patent application EP21153362.5, filed Jan. 25, 2021, entitled “Optimized Acoustic Chirp based on in-vivo BM-delays in human,” which is hereby incorporated herein by reference in its entirety.

### TECHNICAL FIELD

The present invention relates to acoustic chirp stimulus and audiogram systems and methodologies, which advantageously are based on measured in-vivo basilar membrane time delays.

### BACKGROUND ART

It is known in the art to provide auditory stimulus to the ear to evoke an auditory response when testing the hearing of a patient. The magnitude of such an auditory evoked response is typically dependent on the number of neural elements that are activated by the stimulus. As a result, a click stimulus may be utilized that simultaneously outputs a wide range of frequencies with the aim of obtaining larger auditory responses.

However, transduction of sound in human ears is mediated by basilar membrane (BM) waves exhibiting delays that increase with distance from the cochlear base. Consequently, a click stimulus does not cause simultaneous stimulation of the various frequencies along the cochlear due to the travel time through the cochlea, from the basal region (high frequency area) to more apical regions (low frequency area). This causes a blurring of the auditory evoked response.

The concept of the chirp was first applied to auditory electrophysiology by Shore and Nutall. See Shore S E, Nuttall A L. High-synchrony cochlear compound action potentials evoked by rising frequency-swept tone bursts. J Acoust Soc Am. 1985 October; 78(4): 1286-95. (1985), hereby incorporated herein by reference in its entirety. In creating the chirp, the time of individual frequency components within the click are adjusted to compensate for cochlea travel time, resulting in improved temporal synchrony of neural elements and larger auditory responses.

Numerous studies have been performed in order to measure BM delays. Specifically, post mortem studies in various mammalian spices and human have been performed. Another approach to measure BM delays was indirect estimation of BM delays by deriving frequency specific responses from auditory brainstem responses, extracochlear electrocochleography or OAE. Some of these estimates led to assertions that BM delays are much longer in humans than in common experimental animals. See Neely S T, Norton S J, Gorga M P, Jesteadt W. Latency of auditory brain-stem responses and optoacoustic emissions using tone burst stimulus. J. Acoust. Soc. Am. 83:652-656, 1988; Shera C A, Guinan J J, Oxenham A J. Revised estimates of human cochlear tuning from optoacoustic and behavioral measurements. Proc. Natl. Acad. Sci. USA 2002; 99:3318-3323; and Harte J M, Pigasse G, Dau T. Comparison of cochlear delay estimates using optoacoustic emissions and auditory brainstem responses. J Acoust Soc Am. 2009 September; 126(3):1291-301. doi: 10.1121/1.3168508), each of which is hereby incorporated herein by reference in its entirety. Ruggero and Temchin for the first time estimated in vivo BM delays in human cochlea, obtained by correcting postmortem BM data according to the effects of death on BM vibrations in experimental animals. See Ruggero M A, Temchin A N. Similarity of traveling-wave delays in the hearing organs of humans and other tetrapods. J Assoc Res Otolaryngol. 2007 June; 8(2): 153-66, which is hereby incorporated herein by reference in its entirety.

To date, none of the models to create an chirp stimuli have been based on in vivo BM delay measurements of a human. Instead, the delay models have been applied based on a linear description of the mechanical properties of the cochlea (See de Boer E. A cylindrical cochlea model: the bridge between two and three dimensions. Hear Res. 1980 August; 3(2): 109-31), which is hereby incorporated herein by reference in its entirety; tone-burst ABR latencies (See Neely et al., 1988); stimulus-frequency optoacoustic emission latencies (See Shera and Guinan, 2000); derived-band ABR latencies (See Don M, Ponton C W, Eggermont J J, Kwong B. The effects of sensory hearing loss on cochlear filter times estimated from auditory brainstem response latencies. J Acoust Soc Am. 1998 October; 104(4):2280-9. doi: 10.1121/1.423741), which is hereby incorporated herein by reference in its entirety; and acoustically evoked compound action potential (Elberling C, Callø J, Don M. Evaluating auditory brainstem responses to different chirp stimulus at three levels of stimulation. J Acoust Soc Am. 2010 July; 128(1):215-23. doi: 10.1121/1.3397640, which is hereby incorporated herein by reference in its entirety. Additionally, Elberling et al. (2010) found in a large group of normal hearing subjects that the response to various chirp signals is level dependent. This was explained by upward spread of excitation and changes in cochlear-neural delay.

It is now recognized that many cochlear implant candidates nevertheless still have good residual hearing. Unlike conventional hearing aids that just apply an amplified and modified sound signal; a cochlear implant is based on direct electrical stimulation of the acoustic nerve. Typically, a cochlear implant stimulates neural structures in the inner ear electrically in such a way that hearing impressions most similar to normal hearing is obtained.

A normal ear transmits sounds as shown in FIG. 1 through the outer ear 101 to the tympanic membrane (eardrum) 102, which moves the bones of the middle ear 103 (malleus, incus, and stapes) that vibrate the oval window of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolus where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

A typical cochlear prosthesis may include two parts: the audio processor 111 and the implanted stimulator 108. The audio processor 111 typically includes a microphone, a power supply (batteries) for the overall system and a processor that is used to perform signal processing of the acoustic signal to extract the stimulation parameters. The audio processor 111 may be an external behind-the-ear (BTE-) device, may be a single unit that integrates the processor, battery pack and coil or may be implantable.

The stimulator 108 generates the stimulation patterns (based on the extracted audio information) that is sent through an electrode lead 109 to an implanted electrode array 110. Typically, this electrode array 110 includes multiple electrodes on its surface that provide selective stimulation of the cochlea 104. For example, each electrode of the cochlear implant is often stimulated with signals within an assigned frequency band based on the organization of the inner ear, a so-called stimulation channel. The assigned frequency band of an electrode is typically based on its placement within the cochlea, with electrodes closer to the base of the cochlea generally corresponding to higher frequency bands. Electrodes as used for describing the invention and throughout the entire description refer to physical electrode contacts as well as virtual electrode contacts, irrespective of whether they are used for stimulation or measurement. Thus, in the following, electrode refers to both, physical electrode contact 112 and virtual electrode contact.

A stimulation channel corresponds either to a physical electrode contact 112, i.e. an electrode contact that is physically present at a particular location, on the electrode array 110 (physical stimulation channel) or a virtual electrode (virtual stimulation channel) created by stimulating a pair of adjacent physical electrode contacts 112 synchronously with some fixed ratio of currents. In the case of stimulating a pair of adjacent physical electrode contacts 112, the electric fields of the pair of adjacent physical electrode contacts superimpose and the (aforementioned) ratio determines the strength of the two electric fields relative to each other and consequently the superimposed total electric field that in turn can be represented through a virtual electrode located in between the two physical electrode contacts 112. A pair of physical electrode contacts 112 are defined to be adjacent, for as long as the superimposed total electric field can be represented through an electric field that would be generated by stimulating a single virtual electrode contact instead. For example, a ratio of 0.5 determines that the same current is applied to both physical electrode contacts 112 and in effect the total electric field approximately corresponds to one that would be generated by a virtual electrode being located approximately in the middle between the two physical electrode contacts on the electrode array 110. A ratio of 0.3 determines that on one of the two physical electrode contacts 30% of the total delivered current is applied and 70% on the respective other physical electrode contact. In effect, this corresponds to a virtual electrode being located in between the two physical electrode contacts but (normally) closer to the contact where 70% of the current is applied.

The exact same as for stimulation applies for measurements. Here, rather than the stimulation signal, the measurement signal recorded at adjacent physical electrode contacts 112 is weighted. The weighting factor represents the virtual electrode contact, that as stated above, represents an electrode contact in-between two physical electrode contacts. The such weighted measurement signal can then be associated with a specific (acoustic) frequency, for example the frequency to be tested, with e.g. the Greenwood-function as mapping and/or computer tomographic image or both.

The connection between a BTE audio processor and stimulator is usually established by means of a radio frequency (RF-) link. Note that via the RF-link both stimulation energy and stimulation information are conveyed. Typically, digital data transfer protocols employing bit rates of some hundreds of kBit/s are used.

For optimal hearing performance, repeated adjustment of strategy-related map parameters, that are used for programming a cochlear implant prosthesis system to the specifications and needs of its user may be performed from time to time. This is especially true for the electric dynamic range (DR), which is defined by the maximum comfortable loudness (MCL) and threshold (THR)-charge level for each electrode, and which influences performance strongly. The MCL indicates the level at which perceived sound is loud but comfortable; while the THR typically indicates the threshold of hearing. Typically, an increase in MCL or M-level stimulation amplitudes has been found during the first year post implantation, while at the same time electrode impedance values (EIVs) decrease. Usually, stabilization of stimulation levels and EIVs occurs after approximately three months.

In clinical routine, the map parameters are usually adjusted in several sessions by an audiologist on a fixed schedule. Additional visits may be necessary if a CI patient complains about dysfunction or non-optimal functionality of the CI system.

Sometimes it is difficult to measure audiograms in patients implanted with a cochlear implant with residual hearing, especially in children. Therefore, to have an objective method to estimate the audiogram may be useful. The need to obtain an audiogram is necessary in the programming of the processor, it contains i.e. information on the cut-off frequency deciding on what portion of the cochlea is stimulated acoustically and what portion of stimulated electrically, or combined, selecting what electrodes are activated or deactivated, change of frequency allocation or AGC parameters.

In addition to the need for an objective audiogram test method for cochlear implant users with residual hearing, it is important to keep test time at a minimum. Accurate compensation for cochlear travel time resulting in increased temporal synchrony of neural elements could therefore be one way of increasing response amplitude and thereby increase measurement sensitivity, i.e. increase accuracy, allows for lower stimulation levels and in addition shorten test time.

### SUMMARY OF THE EMBODIMENTS

In accordance with an embodiment of the invention, a method of providing an acoustic stimulus for a human subject so as to evoke an auditory response is presented. The method includes creating a chirp signal, wherein creating the chirp signal includes adding a plurality of frequency signals, each frequency signal delayed within the chirp signal based on its associated frequency specific basilar membrane delay determined as a function of measured in vivo frequency specific basilar membrane delays.

In accordance with related embodiment of the invention, the method may further include acoustically providing the chirp signal to the human subject so as to evoke an auditory response. The human subject may have an implanted cochlear implant, and the auditory response is measured using intracochlear.

In accordance with further related embodiments of the invention, the method may further include measuring the in vivo frequency specific basilar membrane delays by, at least in part, either: measuring the in vivo frequency specific basilar membrane delays of a plurality of people; or measuring the in vivo frequency specific basilar membrane delays of the human subject. Measuring the frequency specific basilar membrane delays may include taking measurements using intracochlear electrocochleography. Taking measurements using intracochlear electrocochleography may include providing acoustic frequency tone stimulation, and measuring a cochlear microphonic (CM) response via an electrode of an implanted cochlear implant. The location of each electrode may be determined based on computed tomographic imaging; whereby the Greenwood function may be used to derive a characteristic frequency associated with each electrode, and whereby the measured electrode is the electrode having the characteristic frequency that best matches the acoustic frequency of the tone stimulation provided.

In accordance with yet further related embodiments of the invention, the function of the measured frequency specific basilar membrane delays may include a polynomial or exponential function estimation. A lower frequency signal frequency in the plurality of frequency signals may be delayed less than a higher frequency. Creating the chirp signal may include setting each frequency signal in the chirp signal to the same amplitude and/or loudness perception level of the human subject.

In accordance with another related embodiment of the invention, the human subject has an implanted cochlear implant. The method may further include measuring the in vivo frequency specific basilar membrane delays of the human subject using, at least in part, intracochlear electrocochleography. The chirp signal to acoustically provided to the human subject so as to evoke to evoke an auditory response. The response is measured using intracochlear electrocochleography.

In accordance with another embodiment of the invention, a system for providing an acoustic stimulus for a human subject so as to evoke an auditory response is presented. The system includes a controller configured to create a chirp signal, wherein creating the chirp signal includes adding a plurality of frequency signals, each frequency signal delayed within the chirp signal based on its associated frequency specific basilar membrane delay determined as a function of measured in vivo frequency specific basilar membrane delays.

In accordance with related embodiments of the invention, the system may further include a transducer. The controller may be configured to provide the chirp signal to the transducer so as to evoke an auditory response in the human subject. The system may further include a cochlear implant for implantation in the human subject, wherein the auditory response is measured using intracochlear electrocochleography.

In accordance with further related embodiments of the invention, the in vivo frequency specific basilar membrane delays may be based on measured in vivo frequency specific basilar membrane delays from a plurality of people.

In accordance with still further related embodiments of the invention, the system may further include a transducer, and a cochlear implant for implantation in the human subject. The controller is configured to: provide acoustic frequency tone stimulation to the transducer so as to evoke auditory responses in the human subject; determine the in vivo frequency specific basilar membrane delays of the human subject from the auditory responses from the tone stimulation measured using intracochlear electrocochleography; and provide the chirp signal to the transducer so as to evoke an auditory response from the chirp signal in the human subject. The controller may be configured to determine the location of each electrode based on computed tomographic imaging, use the Greenwood function to derive a characteristic frequency associated with each electrode; and measure the cochlear microphonic (CM) response on the electrode that has the characteristic frequency that best matches the acoustic frequency of the tone stimulation provided.

In further related embodiments of the invention, the function of the measured frequency specific basilar membrane delays may include using a polynomial or exponential function estimation. The controller may be configured to set each frequency signal in the chirp signal to the same amplitude and/or loudness perception level of the human subject.

In accordance with another embodiment of the invention, a method of determining frequency specific basilar membrane delay of a human subject having an implanted cochlear implant is provided. The method includes providing acoustic frequency tone stimulation to the human subject so as to evoke auditory responses in the human subject, the acoustic tone frequency stimulations including acoustic tone pips at a plurality of frequencies. The cochlear microphonic (CM) responses to the acoustic frequency tone stimulation are measured using intracochlear electrocochleography. The in vivo frequency specific basilar membrane delays of the human subject are determined for each of the plurality of the frequencies.

In accordance with related embodiments of the invention, the method may further include creating a chirp signal wherein the frequencies in the chirp signal are delayed in time by their corresponding frequency specific basilar membrane delay. The chirp signal may be acoustically provided to a human subject so as to evoke an auditory response.

In accordance with further related embodiments of the invention, the method may include deriving the location of each electrode based on computed tomographic imaging; and using the Greenwood function to derive a characteristic frequency associated with each electrode. The cochlear microphonic (CM) response is measured using the electrode that has the characteristic frequency that best matches the acoustic frequency of the tone stimulation provided. The method may include includes using a polynomial or exponential function estimation to determine the in vivo frequency specific basilar membrane delays of the human subject for each of the plurality of the frequencies.

In accordance with another embodiment of the invention, a system for determining frequency specific basilar membrane delay of a human subject having an implanted cochlear implant is provided. The system includes a controller configured to: provide acoustic frequency tone stimulation to the human subject via a transducer so as to evoke auditory responses in the human subject, the acoustic tone frequency stimulations including acoustic tone pips at a plurality of frequencies; measure the cochlear microphonic (CM) responses to the acoustic frequency tone stimulation using intracochlear electrocochleography; and determine in vivo frequency specific basilar membrane delays of the human subject for each of the plurality of the frequencies.

In accordance with related embodiments of the invention, the controller may be further configured to create a chirp signal wherein the frequencies in the chirp signal are delayed in time to compensate their corresponding frequency specific basilar membrane delay. The controller may be further configured to acoustically provide the chirp signal to a human subject via the transducer so as to evoke an auditory response.

In accordance with still further embodiments of the invention, the controller may be further configured to: derive the location of each electrode based on computed tomographic imaging; use the Greenwood function to derive a characteristic frequency associated with each electrode; and measure the cochlear microphonic (CM) response using the electrode that has the characteristic frequency that best matches the acoustic frequency of the tone stimulation provided. The controller may be further configured to determine the in vivo frequency specific basilar membrane delays of the human subject for each of the plurality of the frequencies includes using a polynomial or exponential function estimation.

In accordance with another embodiment of the invention, a method of generating an audiogram of a human subject with an implanted cochlear implant based on objective measurements is provided. The cochlear implant includes an electrode array that includes a plurality of electrodes, each electrode associated with a characteristic frequency. The method includes acoustically stimulating the subject with a chirp signal so as to evoke auditory responses in the human subject. The chirp signal includes a plurality of frequency signals, each frequency signal delayed within the chirp signal based on its associated frequency specific basilar membrane delay determined as a function of measured in vivo frequency specific basilar membrane delays. Evoked auditory responses on one or more of the electrodes are measured. Auditory thresholds of the human subject are determined based on the evoked auditory responses. An audiogram of the human subject based on the measured auditory thresholds is determined.

In accordance with related embodiments of the invention, the evoked auditory responses may be measured using intracochlear electrocochleography. The auditory response may be a cochlear microphonic (CM) response (hair cell potential), an auditory nerve neurophonic (ANN) response, or combinations thereof.

In accordance with further related embodiments of the invention, the location of each electrode in the cochlea may be derived based on computed tomographic imaging. The Greenwood function may be used to derive the characteristic frequency associated with each electrode.

In accordance with further related embodiments of the invention, measuring and determining may include the method, wherein measuring and determining includes determining if the chirp signal causes a response on each electrode. If there is no response on any given electrode, reconstruct the chirp signal by increasing the frequency associated with any electrode with no response; acoustically stimulate the subject with the reconstructed chirp signal; and repeat determining, with the reconstructed chirp signal. If responses are measured on all frequencies: reconstruct the chirp signal by decreasing each frequency signal in the chirp; acoustically stimulate the subject with the reconstructed chirp signal so as to evoke auditory responses in the human subject; save an auditory threshold when an electrode no longer provides a response to the chirp signal; determine if the chirp signal causes a response on any electrode; and repeat reconstructing, acoustically stimulating, saving and determining until no response is recorded on any electrode.

In still further related embodiments of the invention, the method may further include modifying fitting parameters of the cochlear implant based on the audiogram. The in vivo frequency specific basilar membrane delays may be measured based, at least in part, by either: measuring the in vivo frequency specific basilar membrane delays of a plurality of people; or measuring the in vivo frequency specific basilar membrane delays of the human subject. Taking measurements using intracochlear electrocochleography may include providing acoustic frequency tone stimulation, and measuring a cochlear microphonic (CM) response via an electrode of an implanted cochlear implant.

In accordance with another embodiment of the invention; a system for generating an audiogram of a human subject with an implanted cochlear implant based on objective measurements is provided. The cochlear implant includes an electrode array that includes a plurality of electrodes, each electrode associated with a characteristic frequency. The system includes a transducer. A controller is configured to create a chirp signal, wherein creating the chirp signal includes adding a plurality of frequency signals, each frequency signal delayed within the chirp signal based on its associated frequency specific basilar membrane delay determined as a function of measured in vivo frequency specific basilar membrane delays. The controller is further configured to: provide the chirp signal to the transducer so as to evoke an auditory response in the human subject; measure the evoked auditory responses on one or more of the electrodes; determine auditory thresholds of the human subject based on the evoked auditory responses; and create an audiogram of the human subject based on the measured auditory thresholds.

In accordance with related embodiments of the invention, the evoked auditory responses are measured using intracochlear electrocochleography. The auditory response may be one of a cochlear microphonic (CM) response (hair cell potential), an auditory nerve neurophonic (ANN) response, or combinations thereof. The controller may be further configured to derive a location of each electrode in the cochlea based on a computed tomographic image; and use the Greenwood function to derive the characteristic frequency associated with each electrode.

In still further related embodiments of the invention, in measuring and determining, the controller may further be configured as follows. Determine if the chirp signal causes a response on each electrode. If there is no response on any given electrode: reconstruct the chirp signal by increasing the frequency associated with any electrode with no response, acoustically stimulate the subject with the reconstructed chirp signal; and repeat determining, with the reconstructed chirp signal. If responses are measured on all frequencies: reconstruct the chirp signal by decreasing each frequency signal in the chirp; acoustically stimulate the subject with the reconstructed chirp; save an auditory threshold when an electrode no longer provides a response; determine if the chirp signal causes a response on any electrode; and repeat reconstructing, acoustically stimulating, saving, and determining until no response is recorded on any electrode.

In yet further related embodiments of the invention, the controller may be configured to modify fitting parameters of the cochlear implant based on the audiogram. The controller may be configured to measure the in vivo frequency specific basilar membrane delays, at least in part, by either: measuring the in vivo frequency specific basilar membrane delays of a plurality of people; or measuring the in vivo frequency specific basilar membrane delays of the human subject. When taking measurements, the controller may be configured to use intracochlear electrocochleography, wherein the controller is configured to provide acoustic frequency tone stimulation, and measure a cochlear microphonic (CM) response via an electrode of the implanted cochlear implant.

In accordance with another embodiment of the invention, a system for generating an audiogram of a human subject with an implanted cochlear implant based on objective measurements is provided. The cochlear implant includes an electrode array that includes a plurality of electrodes, each electrode associated with a characteristic frequency. The system includes: means for acoustically stimulating a the subject with a chirp signal so as to evoke auditory responses in the human subject, the chirp signal including a plurality of frequency signals, each frequency signal delayed within the chirp signal based on its associated frequency specific basilar membrane delay determined as a function of measured in vivo frequency specific basilar membrane delays; means for measuring the evoked auditory responses on one or more of the electrodes; means for determining auditory thresholds of the human subject based on the evoked auditory responses; and means for creating an audiogram of the human subject based on the measured auditory thresholds.

In related embodiments of the invention, the evoked auditory responses may be measured using intracochlear electrocochleography, and the response is at least one of a cochlear microphonic (CM) response (hair cell potential), an auditory nerve neurophonic (ANN) response, or combinations thereof. The system may further include means for deriving a location of each electrode in the cochlea based on computed tomographic imaging; and means for using the Greenwood function to derive the characteristic frequency associated with each electrode. The system may further include means for modifying fitting parameters of the cochlear implant based on the audiogram.

### BRIEF DESCRIPTION OF THE DRAWINGS

The foregoing features of embodiments will be more readily understood by reference to the following detailed description, taken with reference to the accompanying drawings, in which:

FIG. 1 shows a conventional cochlear implant system, in accordance with an embodiment of the invention;

FIG. 2 shows a flow chart for providing an acoustic stimulus for a human subject so as to evoke an auditory response, in accordance with an embodiment of the invention;

FIG. 3 shows a schematic of an exemplary system for measuring and determining the frequency specific BM delays using intracochlear electrocochleography, in accordance with an embodiment of the invention;

FIG. 4 shows an example of intracochlear ECochG recordings, in accordance with an embodiment of the invention;

, FIG. 5 shows an example of a fitting based on measured latencies of in vivo frequency specific basilar membrane (BM) delays determined for frequencies 250, 500, 1000, 2000, 4000 Hz using a latency function, in accordance with an embodiment of the invention;

FIG. 6A shows single frequency signals at the determined latencies that may be used to create a chirp signal, FIG. 6B shows a resulting chirp signal created by adding the frequency signals shown in FIG. 6A, and FIG. 6C shows a resulting a chirp signal based on a fitted frequency range of 250-4010 Hz with linear increase of 20 Hz, in accordance with various embodiments of the invention;

FIG. 7 shows a flow chart for obtaining an audiogram of a human subject with an implanted cochlear implant based on objective measurements, in accordance with an illustrative embodiment of the invention; and

FIG. 8 shows a flowchart of a process for implementing cochlear implant delays, in accordance with an embodiment of the invention.

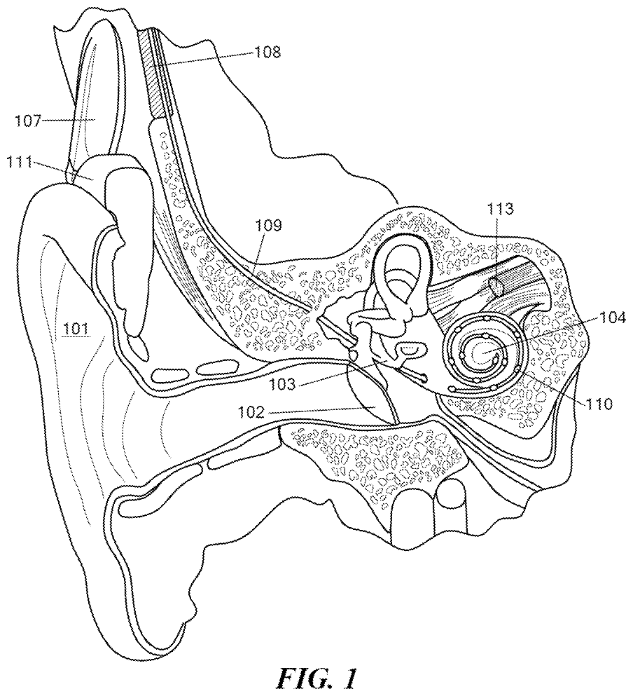

### DETAILED DESCRIPTION OF SPECIFIC EMBODIMENTS

In illustrative embodiments, a system and methodology is provided in which in vivo frequency specific basilar membrane (BM) delays for a plurality of frequencies is measured and determined. The in vivo frequency specific basilar membrane (BM) delays may advantageously be used to create a chirp signal in which frequency signals are delayed within the chirp signal based on its associated frequency specific basilar membrane delay, resulting in improved temporal synchrony of neural elements and larger auditory responses. The chirp signal may be utilized, for example, to acoustically stimulate a cochlear implant subject, whereupon, using intracochlear electrocochleography an audiogram based on objective measurement of residual hearing can be obtained. Details are described below.

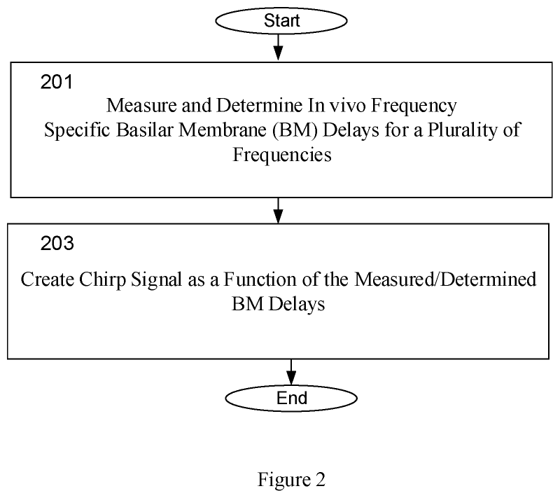

FIG. 2 is a flow diagram of a methodology for providing an acoustic stimulus for a human subject so as to evoke an auditory response, in accordance with an embodiment of the invention. In vivo frequency specific basilar membrane (BM) delays for a plurality of frequencies are measured and determined, step 201. A chirp signal is then created wherein each frequency signal is delayed within the chirp signal based on its associated frequency specific basilar membrane delay determined as a function of the measured/determined in vivo frequency specific BM delays. The chirp stimuli are designed to compensate for the time delay in the auditory periphery in an attempt to increase the temporal synchrony between the neural elements that normally are asynchronously activated by a brief stimulus such as a click. Furthermore, the use of in vivo frequency specific BM delays in creating the chirp signal results in improved temporal synchrony of neural elements and larger auditory responses compared to conventional use of frequency specific BM delays measured/determined ex vivo.

Measuring the in vivo frequency specific basilar membrane delays at step 201 may include making measurements of in vivo frequency specific BM delays of a plurality of people. Various statistical methodologies, as known in the art, may then be utilized in creating the chirp signal. For example, the mean of the determined in vivo frequency specific BM delays may be used to create the chirp signal at step 203. The resulting chirp signal may then be generally used across a wide range of human subjects, preferably as an initial chirp signal.

Alternatively, the determined in vivo frequency specific basilar membrane delays at step 201 may be based on measurements of in vivo frequency specific basilar membrane delays of a specific cochlear implant user (i.e., a patient specific measurement). The chirp signal created at step 203 may then be utilized in further testing of the cochlear implant user. Determining patient-specific in vivo frequency specific basilar membrane delays may result in more accurate chirp signals for the subject cochlear implant user as opposed to using, for example, a mean of such values taken across a wide range of human subjects.

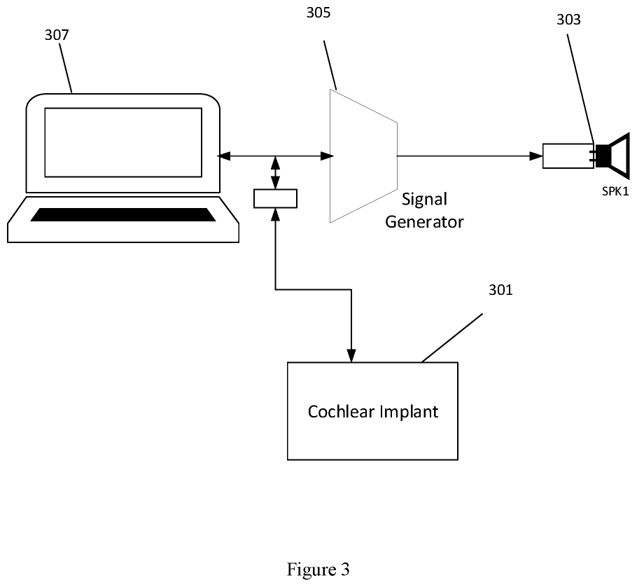

Measuring and determining the frequency specific BM delays at step 201 may be achieved using intracochlear electrocochleography. FIG. 3 shows a schematic of an exemplary system for measuring and determining the frequency specific BM delays using intracochlear electrocochleography, in accordance with an embodiment of the invention. Intracochlear acoustically evoked potentials are recorded from the electrodes of a cochlear implant 301 of a patient inserted in the scala tympani. Acoustic stimulus is presented to the ear canal using a transducer 303, which may be inserted into the ear. The inserts may be connected to a signal generator 305 that creates the acoustic signal. A controller 307 may include software for controlling the system. The controller 307 may be a PC communicating with an interface unit that is connected to the cochlear implant 301 via an external coil. When recording is initiated, the controller 307 may trigger the signal generator 305 which then acoustically stimulates the patient with the chirp signal. Responses from the various electrodes of the cochlear implant 301 may then be recorded. Based on the response, controller 307 may determine frequency specific BM delays, and for example, create a chirp signal that may be used for further acoustic tests.

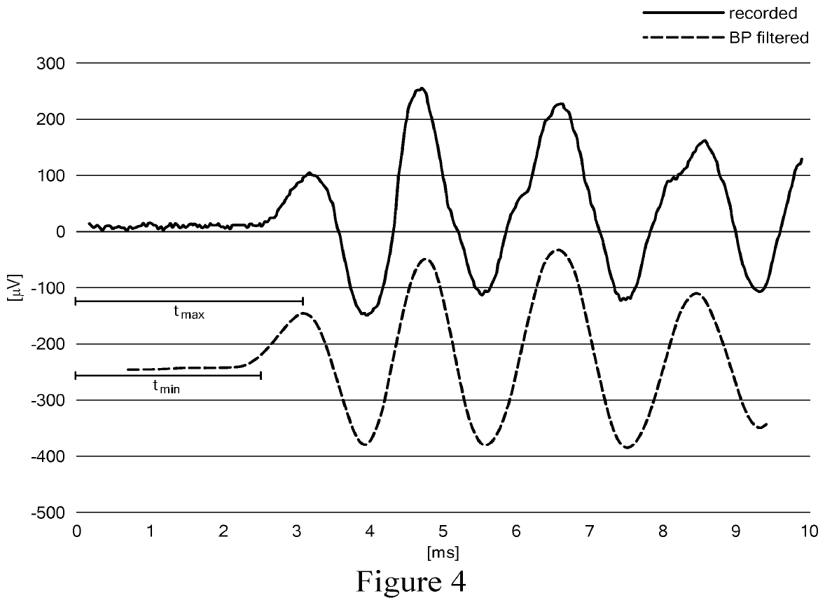

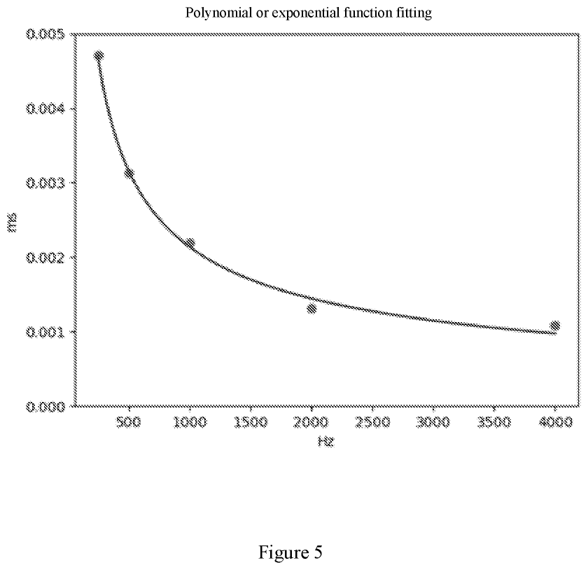

Taking measurements using intracochlear electrocochleography may include providing acoustic frequency tone stimulation, and measuring a cochlear microphonic (CM) response via an electrode of an implanted cochlear implant. The cochlear microphonic (CM) in intracochlear electrocochleography is an alternating current that mirrors the waveform of the acoustic stimulus. It is dominated by the receptor potentials of the outer hair cells of the organ of Corti. Since the CM is proportional to the displacement of the BM, the latency may be measured by illustratively, the 1st peak of the CM or more commonly, the time it takes CM to reached 10% of the maximum amplitude. FIG. 4 shows an example of intracochlear ECochG recordings (black line) and the latency when the CM reached 10% of the maximum amplitude t_{10%} and the latency at the 1^{st} maximum peak tₘₐₓ, in accordance with an embodiment of the invention. The grey line is the BP filtered signal. The stimulus used was a 500 Hz tone pip applied at 0 ms. Other suitable methods or definitions to determine latency can be equally used, e.g. the latency may be when the CM reached 20% of the maximum amplitude t_{20%}. In a further embodiment, the latency may be calculated using a cross-correlation function. The calculation may determine the cross-correlation peak or determine the delay as the minimum delay of the cross-correlation-function exceeding a predetermined threshold. The cross-correlation-function may be normalized, and the predetermined threshold may be 0.75 or 0.9. In one embodiment, the cross-correlation-function from the digitally sampled measurement signal rₖ may be:

$${m}_{d} = \frac{{\vert {\sum }_{k = 0}^{L - 1} {r}_{d + k} \cdot {r}_{d + k + D}^{*} \vert}^{2}}{{( {\sum }_{k = 0}^{L - 1} {\vert {r}_{d + k + D} \vert}^{2} )}^{2}}$$

where D is the number of samples for one period of the measurement signal, e.g. in FIG. 4 for the 500 Hz tone pip one period from one zero-crossing with positive slope to the next zero-crossing with positive slope, and L is the length of the cross-correlation window (L>D). The cross-correlation m_{d} varies from 0 to 1 and is independent on absolute levels (normalized). The delay is found to be the sample number d, where m_{d} exceeds a certain threshold. In one embodiment, the threshold exceeds 0.75 or 0.9. The delay is finally determined to be the time calculated through d divided by sample-rate of the measurement signal rₖ. With this embodiment, band-pass filtering of the measurement-signal is only required for higher or lower-order harmonics and to adopt D to the period of the measurement signal whose delay should be determined, i.e. 250 Hz, 500 Hz, etc. and apply it to the measurement signal rₖ. The higher or lower-order harmonics may be filtered out with the help of band-notch filtering or with proper band-pass filtering.

The acoustic stimulus provided when conducting the intracochlear electrocochleography may include acoustic tone pips at various frequencies at stimulation levels up to, without limitation, the maximum comfortable level. For example, 250, 500, 1000, 2000 and 4000 Hz tone pips may be provided, and the response at the electrode associated with the frequency specific region can be measured. More particularly, the location and/or insertion angle of each electrode in the cochlear implant may be determined based on computed tomographic imaging. The Greenwood function may then be used to derive a characteristic frequency associated with each electrode. The response to a certain tone pip may then be measured on the electrode having the characteristic frequency that best matches the acoustic frequency of the tone pip provided.

The determined latency at each of the provided frequencies may then be fit using, without limitation, a polynomial or exponential function estimation. For example, FIG. 5 shows an example of a fitting based on measured latencies of in vivo frequency specific basilar membrane (BM) delays determined for five different frequencies 250, 500, 1000, 2000, 4000 Hz using a latency function estimated by the function (y=kf\hat{ }(−d)), in accordance with an embodiment of the invention. See Don M, Eggermont J J. Analysis of the click-evoked brainstem potentials in man unsing high-pass noise masking. J Acoust Soc Am. 1978 April; 63(4):1084-92. doi: 10.1121/1.381816, which is hereby incorporated herein by reference in its entirety.

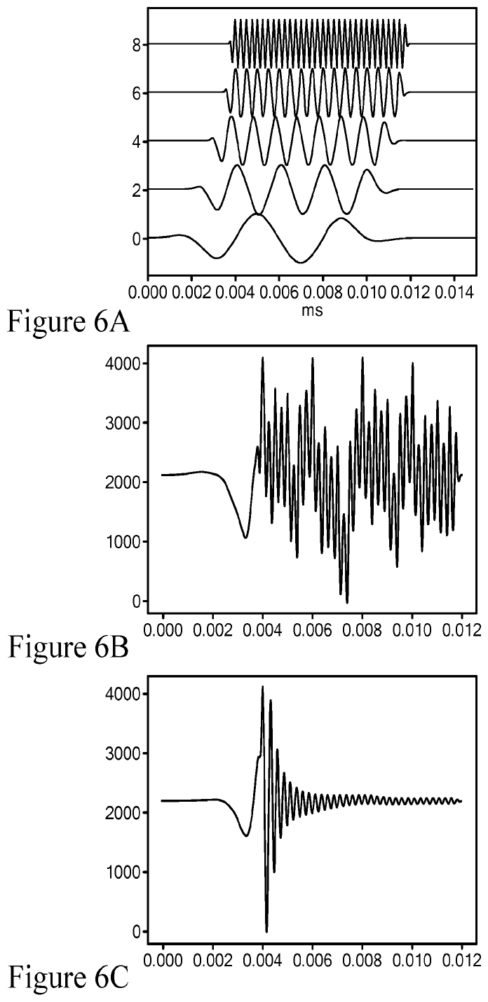

Referring back to step 203 of the FIG. 2, after the in vivo frequency specific basilar membrane (BM) delays across frequencies is determined, a chirp signal is created. FIG. 6A shows single frequency signals at the determined latencies that may be used to create a chirp signal. Each frequency signal in the chirp signal may be set to the same amplitude and/or loudness perception level of the human subject. The resulting chirp signal is then created, without limitation, by adding the frequency signals, as shown in FIG. 6B, in accordance with an embodiment of the invention.

In illustrative embodiments of the invention, the above-described created chirp signal may be used, for example, in subsequent audio tests to acoustically stimulate a human subject so as to evoke an auditory response. Advantageously, objective measurements may be taken. For example, objective measurements based on, without limitation, intracochlear electrocochleography (if the subject has an implanted cochlear implant) or ABR measurements. In various embodiments, the measurements may be used to create an audiogram for the human subject.

As described above, sometimes it is difficult to measure audiograms in patients implanted with a cochlear implant with residual hearing, especially in children. Therefore, to have an objective method to estimate the audiogram may be useful. The data obtained from the audiogram may be used in the fitting of various parameters of the cochlear implant, such as the cut-off frequency deciding on what portion of the cochlea is stimulated acoustically and what portion of the cochlear is stimulated electrically, or what portion of the cochlear is stimulated with both electrical and acoustical stimulation combined, or selecting what electrodes are activated or deactivated, or changes of frequency allocation assigned to stimulation channels or AGC parameters.

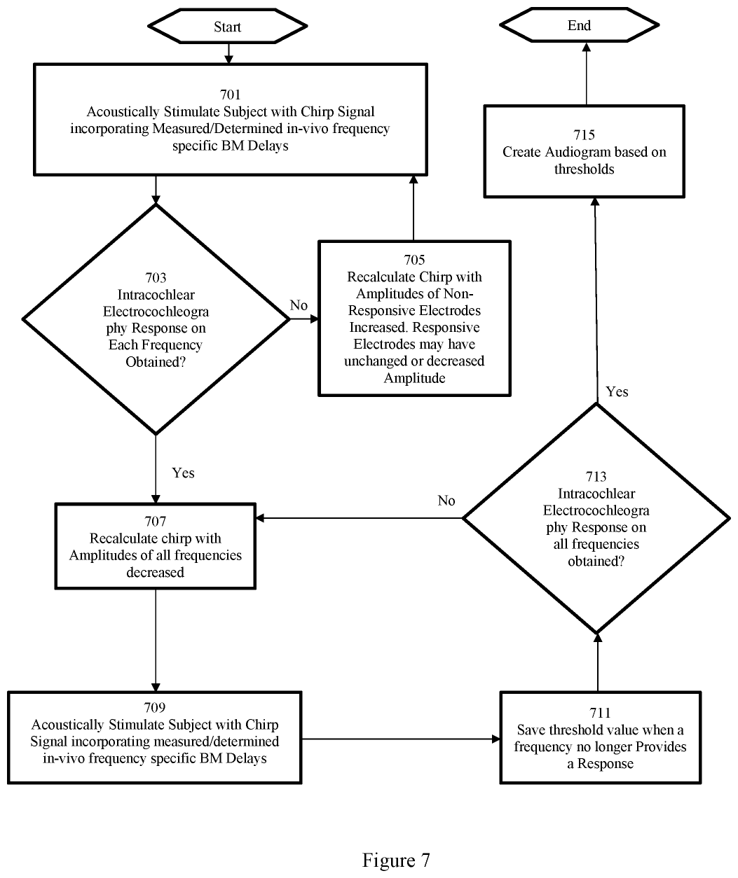

FIG. 7 is a flow chart of a process of obtaining an audiogram of a human subject with an implanted cochlear implant based on objective measurements, in accordance with an illustrative embodiment of the invention. The process may be performed by, without limitation, the system depicted in FIG. 3.

At step 701, acoustic stimulation is presented to the subject, using a chirp signal that compensates the measured/determined in vivo frequency specific basilar membrane (BM) delays, as described above. This chirp signal advantageously maximizes the temporal synchrony between the neural elements within the cochlea, and thereby increasing response amplitudes and thereby increase measurement sensitivity, i.e. increase accuracy, allows for lower stimulation levels and in addition shorten test time.

Initially, the amplitudes of the individual frequencies may be set to a predetermined value that may be, for example, lower than, or close to, the amplitude expected to cause evoked responses.

Intracochlear electrocochleograph may then be used to check if a response on each electrode is obtained, step 703. The response may be, without limitation, the cochlear microphonic (CM) response (hair cell potential) or the auditory nerve neurophonic (ANN) response. If a response is not found on a particular frequency that is being tested (associated with an electrode), the chirp signal is recalculated with the amplitude of that frequency and associated with that electrode increased, step 705. For example, the amplitude for that frequency may be increased, without limitation, by 5 dB or 10 dB.

Upon receiving a response on all frequencies (each associated with an electrode), each of the individual frequencies in the chirp signal are decreased (e.g., by 5 dB), step 707, and the subject is acoustically stimulated with the such re-calculated chirp, step 709. The amplitude when an measurement response for a frequency no longer detectable is saved as threshold for that amplitude, step 711. Step 705 is repeated until no responses are observed on all frequencies, i.e., threshold amplitudes for all tested frequencies have been obtained, step 713. The audiogram (showing the measured thresholds) can then be created, step 715. To increase accuracy of measured threshold amplitudes, the above-described process may be repeated several times.

In the above-described procedure of FIG. 7, the frequency associated with electrode can be determined from postoperative CT scans. From the CT scan, information on the position of each electrode in the cochlea can be determined, along with insertion angle and estimated frequency of excitation, e.g. with the use of Greenwood-function. An association can then be made between the electrode with the closest frequency excitation to a characteristic frequency.

In various embodiments of the invention, obtaining accurate knowledge about the frequency specific time delays within the human cochlea may advantageously help improve audio coding strategies in cochlear implants. It has been shown that hearing impaired patients with various degrees of hearing impairment have different time delays caused by the “artificial” processing programs within their hearing aids or cochlear implant audio processors. See, for example Zirn S, Arndt S, Aschendorff A, Wesarg T. Interaural stimulation timing in single sided deaf cochlear implant users. Hear Res. 2015 October; 328:148-56. doi: 10.1016/j.heares.2015.08.010, which is hereby incorporated herein by reference in its entirety. Interaural stimulation timing mismatches may result in a limitation in the accuracy of temporal binaural processing. By applying frequency specific time delays to cochlear implant audio processors, time delays can be achieved that are equal or close to equal for cochlear implant users in comparison to individuals with normal bilateral hearing. This may be of increased importance as the indication for cochlear implantation continues to expand.

Equal time delays may be particularly important for individuals with single-sided deafness that have a cochlear implant on the non-hearing side. Another special interest group may be individuals with normal or near to normal low frequency hearing preservation after cochlear implantation (Lorens, et al., 2008). Typically, these groups of individuals have a much higher expectation of their hearing performance in comparison to other cochlear implant candidates. These individuals usually reach the ceiling effect for speech tests in quiet, and expect greater improvements with speech in noise test and with spatial hearing abilities.

Note that often it is not sufficient to simply implement BM travelling wave delays into cochlear implant audio processors, an additional delay of 1 ms is also needed. While BM delays represent a delay in the travelling wave, BM vibrations in the respective sensory receptor cells are also stimulated and they release neurotransmitters into the synaptic cleft. The stimulus only excites the auditory nerve fibers after this process. The release of transducers is frequency independent and takes approximately 1 ms. See Temchin A N, Recio-Spinoso A, van Dijk P, Ruggero M A. Wiener kernels of chinchilla auditory-nerve fibers: verification using responses to tones, clicks, and noise and comparison with basilar-membrane vibrations. J Neurophysiol. 2005 June; 93(6):3635-48, which is hereby incorporated by reference herein in its entirety. This frequency independency has previously also been confirmed in human subjects. The first positive peak P1 of the electrically evoked compound action potential occurs 0.6-0.8 ms after the stimulus is elicited and this is achieved independent of the intracochlear place that is being stimulated. See, for example, Polak M, Hodges A V, King J E, Balkany T J. Further prospective findings with compound action potentials from Nucleus 24 cochlear implants. Hear Res. 2004 February; 188(1-2):104-16, which is hereby incorporated by reference herein in its entirety. For electrical stimulation, release of neurotransmitters does not occur and thus this delay should be accounted for in the total time delays.

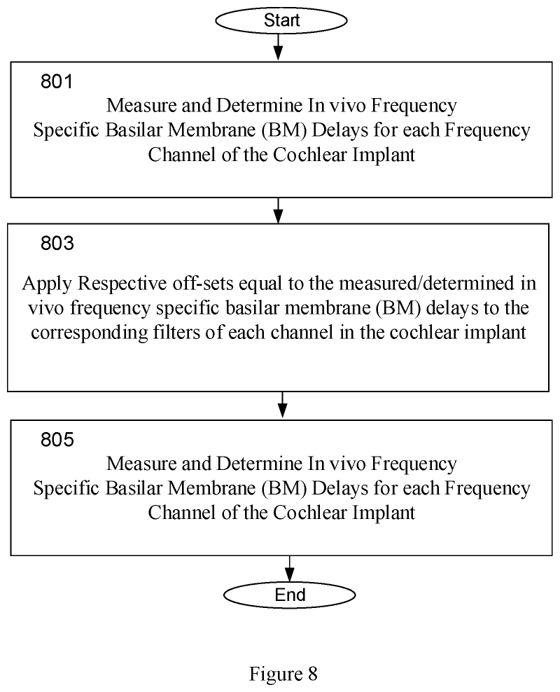

FIG. 8 shows a flowchart of a process for implementing cochlear implant delays. For each frequency channel of the cochlear implant, in vivo frequency specific basilar membrane (BM) delays are measured/determined, as described above, step 801. Respective off-sets equal to the measured/determined in vivo frequency specific basilar membrane (BM) delays are applied to the corresponding filters of each channel in the cochlear implant, step 803. An additional 1 ms offset is added to each offset, step 805. A more accurate interaural time difference (ITD) between the two ears can thus be achieved, an important cue in localizing sound sources.

Embodiments of the invention may be implemented in part in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments also can be implemented in part as a computer program product for use with a computer system—for example, the controller described above. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product). Although various exemplary embodiments of the invention have been disclosed, it should be apparent to those skilled in the art that various changes and modifications can be made which will achieve some of the advantages of the invention without departing from the true scope of the invention.

What is claimed is:

1. A method of providing an acoustic stimulus for a human subject so as to evoke an auditory response, the method comprising:
creating a chirp signal, wherein creating the chirp signal includes adding a plurality of frequency signals, each frequency signal delayed within the chirp signal based on its associated frequency specific basilar membrane delay determined as a function of measured in vivo frequency specific basilar membrane delays.

2. The method according to claim 1, further including acoustically providing the chirp signal to the human subject so as to evoke an auditory response.

3. The method according to claim 2, wherein the human subject has an implanted cochlear implant, and the auditory response is measured using intracochlear electrocochleography.

4. The method according to claim 1, further comprising measuring the in vivo frequency specific basilar membrane delays, at least in part, by either:
measuring the in vivo frequency specific basilar membrane delays of a plurality of people; or
measuring the in vivo frequency specific basilar membrane delays of the human subject.

5. The method according to claim 4, wherein measuring the frequency specific basilar membrane delays includes taking measurements using intracochlear electrocochleography.

6. The method according to claim 5, wherein taking measurements using intracochlear electrocochleography includes providing acoustic frequency tone stimulation, and measuring a cochlear microphonic (CM) response via an electrode of an implanted cochlear implant.

7. The method according to claim 6, further including:
deriving the location of each electrode based on computed tomographic imaging;
using the Greenwood function to derive a characteristic frequency associated with each electrode;
and measuring the cochlear microphonic (CM) response on the electrode that has the characteristic frequency that best matches the acoustic frequency of the tone stimulation provided.

8. The method according to claim 1, wherein the function of the measured frequency specific basilar membrane delays includes using a polynomial or exponential function estimation.

9. The method according to claim 1, wherein a lower frequency signal frequency in the plurality of frequency signals is delayed less than a higher frequency.

10. The method according to claim 1, wherein creating the chirp signal includes setting each frequency signal in the chirp signal to the same amplitude and/or loudness perception level of the human subject.

11. The method according to claim 1, wherein the human subject has an implanted cochlear implant, the method further comprising:
measuring the in vivo frequency specific basilar membrane delays of the human subject using, at least in part, intracochlear electrocochleography,
acoustically providing the chirp signal to the human subject so as to evoke to evoke an auditory response; and
measuring the response by using intracochlear electrocochleography.

12. A system for providing an acoustic stimulus for a human subject so as to evoke an auditory response, the system comprising:
a controller configured to create a chirp signal, wherein creating the chirp signal includes adding a plurality of frequency signals, each frequency signal delayed within the chirp signal based on its associated frequency specific basilar membrane delay determined as a function of measured in vivo frequency specific basilar membrane delays.

13. The system according to claim 12, further comprising a transducer,
wherein the controller is configured to provide the chirp signal to the transducer so as to evoke an auditory response in the human subject.

14. The system according to claim 13, further comprising a cochlear implant for implantation in the human subject, wherein the auditory response is measured using intracochlear electrocochleography.

15. The system according to claim 12, wherein the in vivo frequency specific basilar membrane delays are based on measured in vivo frequency specific basilar membrane delays from a plurality of people.

16. The system according to claim 12, further comprising:
a transducer; and a
a cochlear implant for implantation in the human subject,
wherein the controller is configured to:
provide acoustic frequency tone stimulation to the transducer so as to evoke auditory responses,
determine the in vivo frequency specific basilar membrane delays of the human subject from the auditory responses from the tone stimulation measured using intracochlear electrocochleography,
provide the chirp signal to the transducer so as to evoke an auditory response from the chirp signal in the human subject, and
measure the auditory response of the human subject from the chirp signal using intracochlear electrocochleography.

17. The system according to claim 16, where controller is further configured to:
derive the location of each electrode based on computed tomographic imaging;
using the Greenwood function to derive a characteristic frequency associated with each electrode;
and measure the cochlear microphonic (CM) response on the electrode that has the characteristic frequency that best matches the acoustic frequency of the tone stimulation provided.

18. The system according to claim 12, wherein the function of the measured frequency specific basilar membrane delays includes using a polynomial or exponential function estimation.

19. The system according to claim 12, wherein the controller is configured to set each frequency signal in the chirp signal to the same amplitude and/or loudness perception level of the human subject.

20. A method of determining frequency specific basilar membrane delay of a human subject having an implanted cochlear implant, the method comprising:
providing acoustic frequency tone stimulation to the human subject so as to evoke auditory responses in the human subject, the acoustic tone frequency stimulations including acoustic tone pips at a plurality of frequencies;
measuring the cochlear microphonic (CM) responses to the acoustic frequency tone stimulation using intracochlear electrocochleography; and
determining in vivo frequency specific basilar membrane delays of the human subject for each of the plurality of the frequencies.

21. The method according to claim 20, further comprising creating a chirp signal wherein the frequencies in the chirp signal are delayed in time ti compensate their corresponding frequency specific basilar membrane delay.

22. The method according to claim 21, further comprising acoustically providing the chirp signal to a human subject so as to evoke an auditory response.

23. The method according to claim 20, further including:
deriving the location of each electrode based on computed tomographic imaging;
using the Greenwood function to derive a characteristic frequency associated with each electrode; and
wherein measuring the cochlear microphonic (CM) response uses the electrode that has the characteristic frequency that best matches the acoustic frequency of the tone stimulation provided.

24. The method according to claim 23, wherein determining in vivo frequency specific basilar membrane delays of the human subject for each of the plurality of the frequencies includes using a polynomial or exponential function estimation.

25. A system for determining frequency specific basilar membrane delay of a human subject having an implanted cochlear implant, the system comprising:
a controller configured to:
provide acoustic frequency tone stimulation to the human subject via a transducer so as to evoke auditory responses in the human subject, the acoustic tone frequency stimulations including acoustic tone pips at a plurality of frequencies;
measure the cochlear microphonic (CM) responses to the acoustic frequency tone stimulation using intracochlear electrocochleography; and
determine in vivo frequency specific basilar membrane delays of the human subject for each of the plurality of the frequencies.

26. The system according to claim 25, wherein the controller is further configured to create a chirp signal wherein the frequencies in the chirp signal are delayed in time to compensate their corresponding frequency specific basilar membrane delay.

27. The system according to claim 26, wherein the controller is further configured to acoustically provide the chirp signal to a human subject via the transducer so as to evoke an auditory response.

28. The system according to claim 20, wherein the controller is further configured to:
derive the location of each electrode based on computed tomographic imaging;
use the Greenwood function to derive a characteristic frequency associated with each electrode; and
measure the cochlear microphonic (CM) response using the electrode that has the characteristic frequency that best matches the acoustic frequency of the tone stimulation provided.

29. The system according to claim 23, wherein the controller is configured to determine the in vivo frequency specific basilar membrane delays of the human subject for each of the plurality of the frequencies includes using a polynomial or exponential function estimation.
